(19) United States
(12) Patent Application Publication
(10) Pub. No.: US 2023/0047438 A1
(43) Pub. Date: Feb. 16, 2023

(54) Method and system for assessing disease progression

(71) Applicant: Precision Innovative Data LLC dba Innovative Precision Health (IPH), New Castle, DE (US)

(72) Inventors: Mark Gudesblatt, Sayville, NY (US); Brian Zweben, Baldwin, NY (US); Monte Zweben, San Rafael, CA (US); Suryansh Gupta, Bear, DE (US); Avtej Sethi, Patchogue, NY (US)

(21) Appl. No.: 17/877,666

(22) Filed: Jul. 29, 2022

Related U.S. Application Data
(60) Provisional application No. 63/227,156, filed on Jul. 29, 2021.

(51) Int. Cl. G16H 20/00; G16H 80/00; G16H 10/20; G16H 40/67

(52) U.S. Cl. CPC G16H 20/00; G16H 80/00; G16H 10/20; G16H 40/67

(57) ABSTRACT

A system and method for assessing disease progression receives digital health data about patients over a network from a plurality of diagnostic instruments, IoT devices, analytical software, systems, and electronic health records. Electronic Patient Reported Outcome (PRO) questionnaires are created by clinicians and periodically administered to patients on a remote computing device. The PROs and other digital health data are processed, analyzed, and scored in real time. Digital reports including the scores and other health metrics are instantaneously generated providing valuable hidden insights into disease progression and treatment efficacies in real-time. A clinical advisor generates an interactive dashboard comprising comprehensive information about patients and enables doctors to validate their clinical decisions and discover new treatment protocol idea. Reports useful for other industries may also be generated, such as for pharmaceutical companies, insurance companies, medical researchers, and regulatory agencies.

This application claims the benefit of U.S. Provisional Application No. 63/227,156, filed Jul. 29, 2021, which is hereby incorporated by reference.

### BACKGROUND

More than five million Americans are diagnosed with a serious neurological disorder every year. These disorders have a profound impact on physical and mental health. Quality of life is severely deteriorated. People face substantial and sometimes devastating consequences from neurological disorders.

Neurological disease is very difficult to diagnose and treat. Symptoms erratically come and go, and can include depression, reduced brain function, impaired mobility, spasticity, poor balance, fatigue, bladder and bowel dysfunction, slurred speech, cognitive problems, and so much more. Traditionally, doctors have used a combination of patient history and a neurological examination to treat it. But these traditional neurological exams are not sensitive enough, quantitative enough, or easy enough to document.

The current methods of assessing neurologic disease are inadequate and subjective. There is a disconnect between routine care and the clinical approach employed to demonstrate therapy efficacy. Recent improvements in digital instruments have made it possible to quantitatively measure functional impairment across cognition, sleep, pulmonary function, vision, manual dexterity, gait, and more. But single neurological exams are not comprehensive and fail to capture critical nuances in complex conditions. Treatment efficacy can be difficult to determine due to inconsistencies in measurements and reporting. There are no systems and methods for integrating and quantifying results from multiple complex neurological exams, and to do so in a way that provides an objective and comprehensive assessment of neurological diseases and treatment efficacies.

Even worse, lack of understanding from a patient perspective and patient real-world performance, both cross-sectional and longitudinal, complicate matters even further. At best, patient perspectives are collected manually and are not longitudinally tracked or intensively analyzed. Due to the highly complex multivariate nature of neurological diseases, and cumbersome nature of manually collected patient reported outcomes, these patient perspectives provide only superficial, subjective, and extremely narrow insights into how a patient might be responding to treatments. Despite how critically important patient perspectives are, there are no reliable, easy-to-use, customizable systems or methods to obtain and track patient recoded outcomes and use those outcomes to help objectively assess and guild treatment efficacies for neurological diseases.

Thus, there is a need for a method and system for assessing disease progression.

### SUMMARY

A system for assessing disease progression comprises a patient database for storing health data of patients. An ingestion module ingests data from one or more internet connected devices. The internet connected devices are operable to perform a health exam, medical test, or rehabilitative therapy on a patient and provide digital data of the results of the exam, test, or therapy. A processing module is in communication with the ingestion module and patient database. The processing module processes, cleans, and formats the ingested data, and writes it into the patient database. An electronic health record data integrator is in communication with the ingestion module. It connects with a plurality of electronic health record systems and obtains digital health records patients. The electronic health record data integrator also transmits electronic patient reports to the electronic health record systems.

A patient reported outcome (PRO) module is in communication module. It provides an internet-accessible portal that allows clinicians to select and customize electronic patient reported outcome questionnaires via an electronic interface. The PRO module also administers the electronic patient reported outcome questionnaires to a patient on a remote mobile communication device. And, the PRO module receives from the remote mobile communication device the patient's answers to the questionnaire over the internet. A scoring module is in communication with the PRO module and patient database. The scoring module scores the patient reported outcome questionnaires.

A report module is in communication with the patient database. The report module generates electronic patient reports that capture a health state of the patient. The report module displays the reports on an internet-connected computing device of a doctor. The report module is also in communication with the electronic health record data integrator for the transmitting electronic patient reports to the electronic health record systems. An AI advisor module is in communication with the patient database and report module for performing predictive analytics on the health data of patients stored in the patient database.

A clinical advisor module is in communication with the patient database, report module and AI advisor module. The clinical advisor module is for generating an interactive dashboard accessible by an internet-connected computing device of a doctor. In response to requests form the internet-connected computing device of the doctor, the interactive dashboard displays comprehensive patient information representing the health of the patient and progression of disease in the patient over time and in comparison with other similar patients. It also displays the patient reported outcome questionnaire results and reported outcome scores. Additionally, it displays predictive analytics from the AI advisor module to predict the health outcome of a change in protocol, therapy, or medicine in the treatment of the patient's disease. Furthermore, in response to requests representing the selection and interactions with the displayed patient information on the internet-connected computing device of the doctor, the interactive dashboard displays specific patient reported outcomes, and the results of the patient's exam, test, or therapy from the one or more internet connected devices that are operable to perform a health exam, medical test, or rehabilitative therapy on the patient.

### BRIEF DESCRIPTION OF THE DRAWINGS

FIG. 1 shows a system for assessing disease progression.

FIGS. 2A and 2B show exemplary screenshots of a patient reported outcome mobile application.

FIG. 3 shows the elements of an AI advisor module.

FIG. 4 shows an exemplary electronic patient report.

FIG. 5 shows a login screen of an interactive dashboard.

FIG. 6 shows an interactive dashboard for an exemplary patient.

FIG. 7 shows another interactive dashboard screen highlighting patient reported outcomes.

FIG. 8 shows another interactive dashboard displaying the detailed history of a patient reported outcome selected in FIG. 7.

FIG. 9 shows another interactive dashboard detailing test results of a Gait Assessment which was shown in summary form in FIG. 6.

FIG. 10 shows another dashboard of a Cognitive Assessment displayed after being selected from a corresponding tile in FIG. 6 which showed a summary of the Cognitive Assessment.

FIG. 11 shows yet another interactive dashboard which illustrates a dashboard for a clinic.

FIG. 12 shows a method for assessing disease progression.

FIG. 13 shows the architecture of an exemplary mobile communication device.

### DETAILED DESCRIPTION

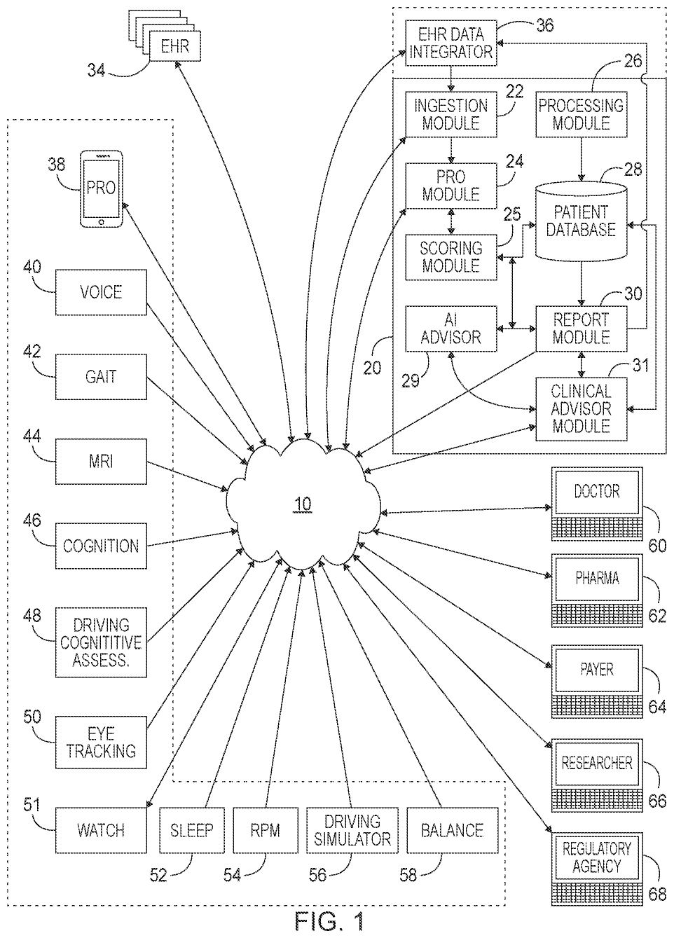

FIG. 1 shows a system for assessing neurological diseases. Briefly, system 20 receives digital health data about patients over a network 10 from at least some of a plurality of instruments, IoT devices, and systems 34, 36, 38, 40, 42, 44, 46, 48, 50, 51, 52, 54, 56, 58 (collectively referred to herein as “instruments” or “devices”). The system 20 processes and scores the data, and creates digital reports received by a doctor 60 on a digital computing device. The reports provide a physician with on-going, clear, quantitative indications of how a patient is responding to particular therapies and treatments over time and in relation to other patient averages enabling the physician to optimize disease treatment.

The system 20 may also comprise an artificial intelligence (AI) advisor 29 to include in the reports personal treatment protocol recommendations that improve patient outcomes. For example, recommendations may include a Disease Modifying Therapy (DMT) or drug that is most likely to impede the progress of neurological disease. These recommendations are made immediately, while the patient is in the office.

The system 20 also includes a clinical advisor module 31 that allows clinicians 60 to securely access and evaluate comprehensive patient profiles from a computer, phone, tablet, and the like. The clinical advisor module 31 is in communication with a patient database 28, a report module 30, and AI advisor 29, and a network 10.

The clinical advisor module 31 generates an interactive dashboard accessible by doctors 60 which displays comprehensive patient information including patient reported outcomes (PROs), digital analytics from devices such as computerized cognitive testing, digital gait analysis, and quantitative MM data, forward looking analytics, and electronic health record data. PROs, digital analytics, devices, electronic health records, and the like will be disclosed in greater detail below.

Doctors may interact with the elements displayed on the dashboard to view various aspects of the patient data with varying level of specificity, and in various ways. For example, graphs showing longitudinal results can show changes in PRO results over time, cyclograms may show gait results, and so forth. In one embodiment, PROs are displayed with scores and benchmark outcomes, for example healthy, average, concern, on the dashboard to provide doctors with an accurate and easy to interpret view of how a disease is progressing for patient. Scores and benchmarks will be disclosed in greater detail below.

The system 20 with clinical advisor module 31 enable doctors to validate their clinical decisions and discover new treatment protocol ideas. Additionally, doctors can run “what-if” scenarios to predict the outcome of a slight change in protocol or medicine for a patient. In this way clinicians can determine the best ongoing treatment for their patients. Clinical Advisor 31 provides an objective, evidence-based view of disease trajectories as well as recommendations for the long-term success of therapies.

The system 20 comprises and ingestion module 22 in communication with network 10. The ingestion module 22 receives data from network connected devices or systems 34, 36, 38, 40, 42, 44, 46, 48, 50, 51, 52, 54, 56, 58.

In communication with the ingestion module 22 is a processing module 26 which processes, cleans, and formats the ingested data for storage in a patient database 28. Processing, cleaning and formatting the ingested data may also include analyzing the ingested data, for example, to identify trends and changes in the data, executing various analyses algorithms and models such as regression analysis, classification, various from predictive analytics including neural networks and machine learning, clustering models, forecasting models, outliers models, time series models, descriptive analysis, exploratory analysis, inferential analysis, predictive analysis, casual analysis, mechanistic analysis, and any other type of analysis known to those having ordinary skill in the art.

Patient database 28 is in communication with the processing module 26 and stores multi-dimensional patient data. The patient database 28 is also in communication with scoring module 25, AI advisor 29, and report module 30. Report module 30 is in communication with network 10, over which electronic reports are delivered to doctor computing device 60 and, in some embodiments, a pharma device 62, payer device 64, researcher device 66, and regulatory agency device 68. The report module 30 is also in communication with EHR Data Integrator 36.

A Patient Reported Outcome (PRO) Module 24 is in communication with network 10, ingestion module 22 and scoring module 25. The PRO Module 24 provides an internet-accessible portal that allows clinicians such as a doctor or a clinic administrator 60 to select and customize electronic patient reported outcome questionnaires via a web interface or equivalent. These questionnaires are administered to patients via network 10 connected PRO device 38, such a computing device like a tablet, via the PRO module 24. A patient answers the questionnaire on device 38. The answers are sent to PRO module 24 of system 28 and ingested 22, processed 26, and stored in patient database 28 with the associated patient ID. Associated with every patient is a unique patient ID.

These digitally administered PROs are scored and reported automatically real time by way of scoring module 25 and reporting module 30. Raw data and calculated metrics are stored in patient database 28 which, in one embodiment, is SOC 2 and HIPAA compliant. Patient data is tracked longitudinally and is accessible via any conventional computing device such as a computer or mobile device like a tablet or phone.

Patient reported outcomes allow clinicians to get a full picture of a patient's environmental, physical, and mental conditions. By way of the reports, these digitized PROs enable clinicians to collect and use patient outcomes for diagnostic purposes. They also give a longitudinal and multi-dimensional view of how a treatment or disease is affecting a patient.

Clinicians can assign PROs based on the patient's disease state. Clinicians can also give patients a general health wellness PRO that may not be associated with a particular disease. There are PRO packet repositories for clinicians to choose from. PROs come grouped in packets depending on their disease state and may be modified and grouped together as collections by the clinician. Examples of disease states for which PROs are available include Alzheimer's disease, Attention Deficit Hyper Disorder (ADHD), Amyotrophic Lateral Sclerosis (ALS), Dementia, Epilepsy and Seizures, Migraines and Headaches, Myasthenia Gravis (MG), Multiple Sclerosis (MS), Neuropathy/Polyneuropathy, Parkinson's Disease (PD), Stroke, Fibromyalgia, Gait Abnormalities, Insomnia and Narcolepsy. Other PROs include questionnaires for anxiety and depression, sleep disturbances, brief illness perception, modified fatigue impact scale, emotional behavioral dyscontrol, and Zarit Burden.

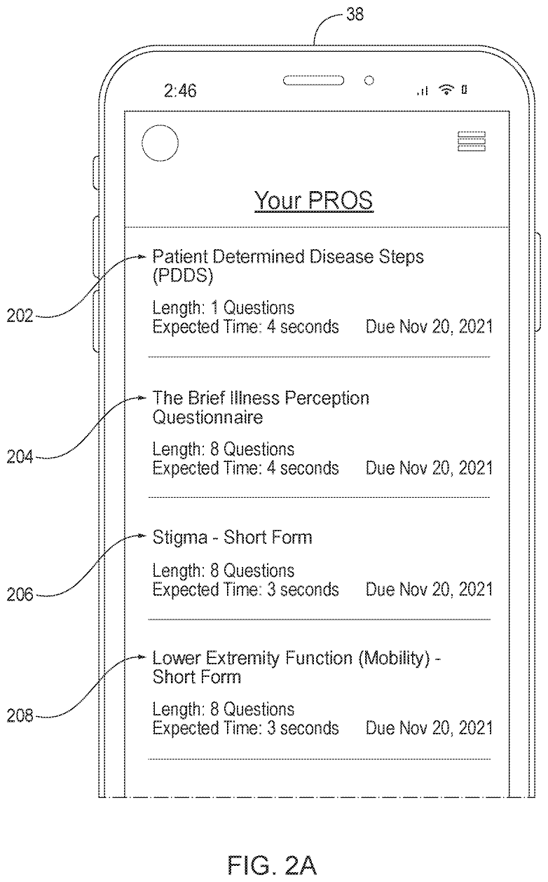

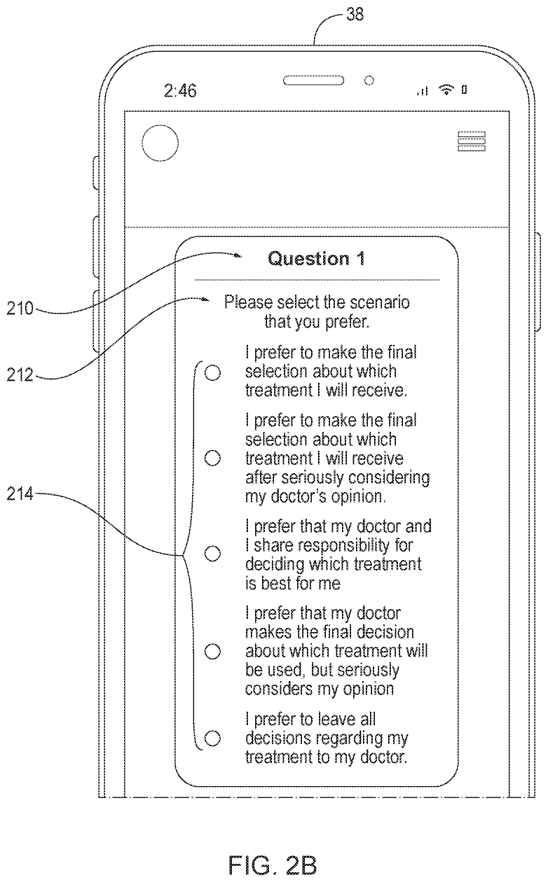

To get a better idea of how the PROs configured and assigned to a particular patient might be administered, FIGS. 2A and 2B show exemplary screenshots of a Patient Reported Outcome mobile application on PRO device 38 of FIG. 1.

In FIG. 2A, displayed to the patient are all of the PROs that the clinician has assigned. In this example there are four (4) PROs, Patient Determined Disease Steps (PDDS) 202, The Brief Illness Perception Questionnaire 204, Stigma—Short Form 206, and Lower Extremity Function (Mobility)—Short Form 208. The may be greater or fewer than four PROs for a patient to complete and the PROs may be different than those shown in the exemplary figure. Underneath each PRO title 202, 204, 206, 208 is information about the PRO, such as the number of questions and expected time to complete the questions, and due date.

When the patient selects a PRO, the PROs questions are displayed to the patient. FIG. 2B shows an exemplary question 210 of one of the exemplary PROs 202, 204, 206, 208. There may be many questions and they will be different depending on the PRO. In the case the patient is asked “Please select the scenario that you prefer” 212 and is given five option to select about final selections of treatment 214. In this example radio buttons with text are displayed for selection by the patient. However, PRO questionnaire screens may have any type of input possible on electronic computing devices such as text input, date, dropdown menus, radio button with text, checkbox with text, photo, multiple choice, multiple choice-scaled question, multiple choice question with picture, voice recording, and so forth.

After the PRO is completed, it is sent to PRO Module 24 of system 20 (see FIG. 1) as disclosed above for ingestion 22, processing 26, scoring 25, and storage 28. In one embodiment the a JSON message is sent from the PRO Device 28 running the PRO application to the system 20. Exemplary code for an exemplary PRO “Multiple Sclerosis Impact Scale (MSIS-29)” is:

This is just one example, and with the above disclosure it can now be appreciated how PROs can be administered to a patient on a mobile device 38 and sent to system 20 for processing and storage.

As disclosed above, the PROs are scored by Scoring Module 25 of FIG. 1. The following shows exemplary code of the Scoring Module 25 for scoring a PRO received from the PRO Module 24 which was sent to system 20 by the PRO device app 38. In this example, the PRO is “The Brief Illness Perception Questionnaire”. Other PROs are possible of course, and they will follow the same format sequences as shown below.

The exemplary received PRO data for “The Brief Illness Perception Questionnaire” received by JSON message from the PRO App 38 and disclosed above into PRO Module 24 is:

{′due_date′: ′2022-06-29 11:59:00′, ′name′: ′The Brief Illness Perception Questionnaire′, ′descrip-
tion′: ′For the following questions, please select the number that best corresponds to your views:′,
′id′: ′32623′, ′duration′: ′6′, ′user_epro_id′: 4052, ′collection_name′: ′General FNS, ′clinicId′: ′2037′,
′patientId′: ′testpatientID′, ′mrnNumber′: ′testpatientMRN′, ′submitDate′: ′submitDate′, ′timeTaken′:
′11s′, ′questionList′: [{′id′: 325, ′epro_id′: ′35′, ′question_type′: ′MULTIPLE_CHOICE′, ′label′: ′How
much does your illness affect your life?′, ′items′: ′[0, 1, 2, 3, 4, 5, 6, 7, 8, 9, 10]′, ′sequence′: ′0′,
′is_compulsory′: ′1′, ′remarks′: ″, ′image′: ″, ′file_url′: ”, ′filename′: ″, ′status′: ′1′, ′created_by′: ′1′,
′created_at′: ′2021-09-22T02:46:43.000000Z′, ′updated_at′: ′2021-09-22T02:47:48.000000Z′, ′an-
swer′: ′4′, ′instruction′: ′0 = No affect at all; 10 = severely affects my life′}, {′id′: 326, ′epro_id′: ′35′,
′question_type′: ′MULTIPLE_CHOICE′, ′label′: ′How long do you think your illness will continue?′,
′items′: ′[0, 1, 2, 3, 4, 5, 6, 7, 8, 9, 10]′, ′sequence′: ′0′, ′is_compulsory′: ′1′, ′remarks′: ″, ′image′: ″,
′file_url′: ″, ′filename′: ”, ′status′: ′1′, ′created_by′: ′1′, ′created_at′: ′2021-09-22T02:47:51.000000Z′,
′updated_at′: ′2021-09-22T02:48:43.000000Z′, ′answer′: ′9′, ′instruction′: ′0 = a very short time; 10
= Forever′}, {′id′: 327, ′epro_id′: ′35′, ′question_type′: ′MULTIPLE_CHOICE′, ′label′: ′How much
control do you feel you have over your illness?′, ′items′: ′[0, 1, 2, 3, 4, 5, 6, 7, 8, 9, 10]′, ′se-
quence′: ′0′, ′is_compulsory′: ′1′, ′remarks′: ″, ′image′: ″, ′file_url′: ″, ′filename′: ″, ′status′: ′1′, ′cre-
ated_by′: ′1′, ′created_at′: ′2021-09-22T02:48:45.000000Z′, ′updated_at′: ′2021-09-
22T02:49:41.000000Z′, ′answer′: ′7′, ′instruction′: ′0 = Absolutely no control; 10 = extreme amount
of control′}, {′id′: 328, ′epro_id′: ′35′, ′question_type′: ′MULTIPLE_CHOICE′, ′label′: ′How much do
you think your treatment can help your illness?′, ′items′: ′[0, 1, 2, 3, 4, 5, 6, 7, 8, 9, 10]′, ′se-
quence′: ′0′, ′is_compulsory′: ′1′, ′remarks′: ″, ′image′: ″, ′file_url′: ″, ′filename′: ″, ′status′: ′1′, ′cre-
ated_by′: ′1′, ′created_at′: ′2021-09-22T02:49:45.000000Z′, ′updated_at′: ′2021-09-
22T02:50:51.000000Z′, ′answer′: ′3′, ′instruction′: ′0 = Not at all; 10 = extremely helpful′}, {′id′: 329,
′epro_id′: ′35′, ′question_type′: ′MULTIPLE_CHOICE′, ′label′: ′How much do you experience symp-
toms from your illness?′, ′items′: ′[0, 1, 2, 3, 4, 5, 6, 7, 8, 9, 10]′, ′sequence′: 0′, ′is_compulsory′:
′1′, ′remarks′: ″, ′image′: ″, ′file_url: ″, ′filename′: ″, ′status′: ′1′, ′created_by′: ′1′ ′created_at′: ′2021-
09-22T02:50:53.000Q00Z′, ′updated_at′: ′2021-09-22T02:52:09.000000Z′, ′answer′: ′10′, ′instruc-
tion′: ′0 = No symptoms at all; 10 = many severe symptoms′}, {′id′: 330, ′epro_id′: ′35′, ′ques-
tion_type′: ′MULTIPLE_CHOICE′, ′label′: ′How concerned are you about your illness?′, ′items′: ′[0,
1, 2, 3, 4, 5, 6, 7, 8, 9, 10]′, ′sequence′: ′0′, ′is_compulsory′: ′1′, ′remarks′: ″, ′image′: ″, ′file_url′: ″,
′filename′: ″, ′status′: ′1′, ′created_by′: ′1′, ′created_at′: ′2021-09-22T02:52:11.000000Z′, ′up-
dated_at′: ′2021-09-22T02:53:47.000000Z′, ′answer′: ′9′, ′instruction′: ′0 = Not at all concerned; 10
= extremely concerned′}, {′id′: 331, ′epro_id′: ′35′, ′question_type′: ′MULTIPLE_CHOICE′, ′label′:
′How well do you feel you understand your illness?′, ′items′: ′[0, 1, 2, 3, 4, 5, 6, 7, 8, 9, 10]′, ′se-
quence′: ′0′, ′is_compulsory′: ′1′, ′remarks′: ″, ′image′: ″, ′file_url′: ″, ′filename′: ″, ′status′: ′1′, ′cre-
ated_by′: ′1′, ′created_at′: ′2021-09-22T02:53:55.000000Z′, ′updated_at′: ′2022-01-
29T03:45:44.000000Z′, ′answer′: ′9′, ′instruction′: ′0 = Dont understand at all; 10 = understand
very clear′}, {′id′: 333, ′epro_id′: ′35′, ′question_type′: ′MULTIPLE_CHOICE′, ′label′: ′How much
does your illness affect you emotionally? (e.g does it make you angry, scared, upset, or de-
pressed?)′, ′items′: ′[0, 1, 2, 3, 4, 5, 6, 7, 8, 9, 10]′, ′sequence′: 0′, ′is_compulsory′: ′1′, ′remarks′: ″,
′image′: ″, ′file_url′: ″, ′filename′: ″, ′status′: ′1′, ′created_by′: ′1′, ′created_at′: ′2021-09-
22T02:54:57.000000Z′, ′updated_at′: ′2021-09-22T02:56:19.000000Z′, ′answer′: ′10′, ′instruction′:
′0 = Not at all affected emotionally; 10 = extremely affected emotionally′}], ′last_pro_submitted′:
True}

The following exemplary code processes and scores the PRO:

```
def BIP(input):
  # The Brief Illness Perception Questionnaire
  questionList = input[“questionList”]
  payload = { }
  patientID = getHashFromPRO2(input)
  for i in range(len(questionList)):
    payload.update({f“Q{i+1}”: questionList[i][“answer”]})
  score, resDict = scoreBIP(payload)
  payload = resDict
  clinicid = clinicIDmap(input[“clinicId”])
  payload.update({f“SCORE”: score})
  payload.update({f“TEST_DATE”: str(datetime.datetime.now( ))})
  payload.update({f“PATIENT_ID”: patientID})
  payload.update({f“CLINIC_ID”: clinicid})
  payload.update({f“MIN”: 0})
  payload.update({f“LOW”: 30})
  payload.update({f“MID”: 40})
  payload.update({f“HIGH”: 50})
  payload.update({f“MAX”: 80})
  payload.update({f“SCORED_POSITIVELY”: False})
  payload.update({f“SUBSCORES”: { }})
  payload.update({f“GRAPH_TYPE”: Enums.GraphTypes.GRADIENT.name})
  print(json.dumps(payload, indent=4))
  payload.update({f“RAW”: input})
  response = collectionUpsert(“PROD_PRO_BRIEF_ILLNESS_PERCEPTION”, payload)
  return response
def scoreBIP(QA):
  rubric = {
    “Q1”: {
      “10”: 10,
      “9”: 9,
      “8”: 8,
      “7”: 7,
      “6”: 6,
      “5”: 5,
      “4”: 4,
      “3”: 3,
      “2”: 2,
      “1”: 1,
      “0”: 0,
    },
    “Q2”: {
      “10”: 10,
      “9”: 9,
      “8”: 8,
      “7”: 7,
      “6”: 6,
      “5”: 5,
      “4”: 4,
      “3”: 3,
      “2”: 2,
      “1”: 1,
      “0”: 0,
    },
    “Q3”: {
      “10”: 0,
      “9”: 1,
      “8”: 2,
      “7”: 3,
      “6”: 4,
      “5”: 5,
      “4”: 6,
      “3”: 7,
      “2”: 8,
      “1”: 9,
      “0”: 10,
    },
    “Q4”: {
      “10”: 0,
      “9”: 1,
      “8”: 2,
      “7”: 3,
      “6”: 4,
      “5”: 5,
      “4”: 6,
      “3”: 7,
      “2”: 8,
      “1”: 9,
      “0”: 10,
    },
    “Q5”: {
      “10”: 10,
      “9”: 9,
      “8”: 8,
      “7”: 7,
      “6”: 6,
      “5”: 5,
      “4”: 4,
      “3”: 3,
      “2”: 2,
      “1”: 1,
      “0”: 0,
    },
    “Q6”: {
      “10”: 10,
      “9”: 9,
      “8”: 8,
      “7”: 7,
      “6”: 6,
      “5”: 5,
      “4”: 4,
      “3”: 3,
      “2”: 2,
      “1”: 1,
      “0”: 0,
    },
    “Q7”: {
      “10”: 0,
      “9”: 1,
      “8”: 2,
      “7”: 3,
      “6”: 4,
      “5”: 5,
      “4”: 6,
      “3”: 7,
      “2”: 8,
      “1”: 9,
      “0”: 10,
    },
    “Q8”: {
      “10”: 10,
      “9”: 9,
      “8”: 8,
      “7”: 7,
      “6”: 6,
      “5”: 5,
      “4”: 4,
      “3”: 3,
      “2”: 2,
      “1”: 1,
      “0”: 0,
    }
  }
  resDict = { }
  total = 0
  for key, val in QA.items( ):
    resDict.update({key: rubric[key][val]})
    total += rubric[key][val]
  return total, resDict
```

And the output after processing and scoring which is stored in patient database 28 is:

```
{
  “Q1”: 4,
  “Q2”: 9,
  “Q3”: 3,
  “Q4”: 7,
  “Q5”: 10,
  “Q6”: 9,
  “Q7”: 1,
  “Q8”: 10,
  “SCORE”: 53,
  “TEST_DATE”: “2022-06-29 16:42:01.725507”,
  “PATIENT_ID”: “15779”,
  “CLINIC_ID”: “2037”,
  “MIN”: 0,
  “LOW”: 30,
  “MID”: 40,
  “HIGH”: 50,
  “MAX”: 80,
  “SCORED_POSITIVELY”: false,
  “SUBSCORES”: { },
  “GRAPH_TYPE”: “GRADIENT”
}
```

As can be seen, in this example, the SCORE for this PRO is 53 which places it between the HIGH and MAX range.

Other PROs may have different specific number values but the processing and scoring methods disclosed above are applicable to all PROs. Code for scoring a multiplicity of different PROs is shown in the Appendix to the Specification, “PRO Functions Scoring Handling Code”.

Turning back to FIG. 1, patient information from Electronic Health Records (EHR) 34 is stored in patient database 28 with the PROs and other health data which will be disclosed below. This builds a multivariate longitudinal and cross-sectional view of patients which can then be generated into interactive reports by Report Module 30.

There are dozens of EHR systems 34. Just a few examples include EPIC, Cerner, AthenaHealth, and Nextgen. There are more than a thousand healthcare providers. In order to provide broadest compatibility with EHR systems 34 used by providers, and to ensure security, an EHR Data Integrator module 36 may be employed to facilitate connecting to various EHR systems 36.

The EHR Data Integrator 36 is also in communication with Report Module 30. In one embodiment, the EHR Data Integrator 36 receives electronic reports from the Report Module 30 and securely transmits them to one or more EHR systems over network 10 for storage as part of the patient's electronic health records.

One example of an EHR Data Integrator is the Redox EHR Integration API by REDOX (https://www.redoxengine.com/). Another example is Mirth Connect which is a cross-platform interface engine used in the healthcare industry (https://www.nextgen.com/products-and-services/integration-engin). Yet another example is Fast Healthcare Interoperability Resources (FHIR) which is a standard for exchanging healthcare information electronically (https://www.fhir.org/). Other examples include web scraping scripts, automating scripts and the like.

Electronic health record data is transferred from the EHR 34 through the EHR Data Integrator 36 autonomously thereby preventing human error and ineffective transfers. The Data Integrator 36 formats and passes the data through to the ingestion module 22. All communications are secured via SSL. No data is stored by the Data Integrator 36.

Once ingested by the Ingestion Module API 22, the data is deidentified, depending on various configurations. Some of the patient health record information that may be removed, in whole or in part, includes name, address, social security number, medical record number, birthdate, and contact information. The patient health information is then stored in the patient database 28.

Electronic medical records are received from the EHR Data integrator 36 in a JSON format. One exemplary JSON medical record is:

```
{
  “Meta”: {
    “DataModel”: “PatientAdmin”,
    “EventType”: “PatientUpdate”,
    “EventDateTime”: “2022-07-25T17:15:27.690Z”,
    “Test”: true,
    “Source”: {
      “ID”: “7ce6f387-c33c-417d-8682-81e83628cbd9”,
      “Name”: “Redox Dev Tools”
    },
    “Destinations”: [
      {
        “ID”: “af394f14-b34a-464f-8d24-895f370af4c9”,
        “Name”: “Redox EMR”
      }
    ],
    “Logs”: [
      {
        “ID”: “d9f5d293-7110-461e-a875-3beb089e79f3”,
        “AttemptID”: “925d1617-2fe0-468c-a14c-f8c04b572c54”
      }
    ],
    “Message”: {
      “ID”: 5565
    },
    “Transmission”: {
      “ID”: 12414
    },
    “FacilityCode”: null
  }
  “Patient”: {
    “Identifiers”: [
      {
        “ID”: “0000000001”,
        “IDType”: “MR”
      },
      {
        “ID”: “e167267c-16c9-4fe3-96ae-9cff5703e90a”,
        “IDType”: “EHRID”
      },
      {
        “ID”: “a1d4ee8aba494ca”,
        “IDType”: “NIST”
      }
    ],
    “Demographics”: {
      “FirstName”: “Timothy”,
      “MiddleName”: “Paul”,
      “LastName”: “Bixby”,
      “DOB”: “2008-01-06”,
      “SSN”: “101-01-0001”,
      “Sex”: “Male”,
      “Race”: “White”,
      “IsHispanic”: null,
      “Religion”: null,
      “MaritalStatus”: “Single”,
      “IsDeceased”: null,
      “DeathDateTime”: null,
      “PhoneNumber”: {
        “Home”: “+18088675301”,
        “Office”: null,
        “Mobile”: null
      },
      “EmailAddresses”: [ ],
      “Language”: “en”,
      “Citizenship”: [ ],
      “Address”: {
        “StreetAddress”: “4435 Victoria Ln”,
        “City”: “Madison”,
        “State”: “WI”,
        “ZIP”: “53719”,
        “County”: “Dane”,
        “Country”: “US”
      }
    },
    “Notes”: [ ],
    “Contacts”: [
      {
        “FirstName”: “Barbara”,
        “MiddleName”: null,
        “LastName”: “Bixby”,
        “Address”: {
          “StreetAddress”: “4762 Hickory Street”,
          “City”: “Monroe”,
          “State”: “WI”,
          “ZIP”: “53566”,
          “County”: “Green”,
          “Country”: “US”
        },
        “PhoneNumber”: {
          “Home”: “+18088675303”,
          “Office”: “+17077543758”,
          “Mobile”: “+19189368865”
        },
        “RelationToPatient”: “Mother”,
        “EmailAddresses”: [
          “barb.bixby@test.net”
        ],
        “Roles”: [
          “Emergency Contact”
        ]
      }
    ],
    “Diagnoses”: [
      {
        “Code”: “R07.0”,
        “Codeset”: “ICD-10”,
        “Name”: “Pain in throat”,
        “Type”: null,
        “DocumentedDateTime”: null
      }
    ],
    “Allergies”: [
      {
        “Code”: “7982”,
        “Codeset”: “RxNorm”,
        “Name”: “Penicillin”,
        “Type”: {
          “Code”: null,
          “Codeset”: null,
          “Name”: null
        },
        “OnsetDateTime”: null,
        “Reaction”: [
          {
            “Code”: “28926001”,
            “Codeset”: “SNOMED CT”,
            “Name”: “Rash”
          },
          {
            “Code”: “247472004”,
            “Codeset”: “SNOMED CT”,
            “Name”: “Hives”
          }
        ],
        “Severity”: {
          “Code”: null,
          “Codeset′′: null,
          “Name”: null
        },
        “Status”: null
      }
    ],
    “PCP”: {
      “NPI”: “4356789876”,
      “ID”: “4356789876”,
      “IDType”: “NPI”,
      “FirstName”: “Pat”,
      “LastName”: “Granite”,
      “Credentials”: [
        “MD”
      ],
      “Address”: {
        “StreetAddress”: “123 Main St.”,
        “City”: “Madison”,
        “State”: “WI”,
        “ZIP”: “53703”,
        “County”: “Dane”,
        “Country”: “USA”
      },
      “EmailAddresses”: [ ],
      “PhoneNumber”: {
        “Office”: “+16085551234”
      },
      “Location”: {
        “Type”: null,
        “Facility”: null,
        “Department”: null,
        “Room”: null
      }
    },
    “Insurances”: [
      {
        “Plan”: {
          “ID”: “31572”,
          IDType”: “Payor ID”,
          “Name”: “HMO Deductible Plan”,
          “Type”: null
        },
        “MemberNumber”: null,
        “Company”: {
          “ID”: “60054”,
          “IDType”: null,
          “Name”: “aetna (60054 0131)”,
          “Address”: {
            “StreetAddress”: “PO Box 14080”
            “City”: “Lexington”,
            “State”: “KY”,
            “ZIP”: “40512-4079”,
            “County”: “Fayette”,
            “Country”: “US”
          },
          “PhoneNumber”: “+18089541123”
        },
        “GroupNumber”: “847025-024-0009”,
        “GroupName”: “Accelerator Labs”,
        “EffectiveDate”: “2015-01-01”,
        “ExpirationDate”: “2020-12-31”,
        “PolicyNumber”: “9140860055”,
        “Priority”: null,
        “AgreementType”: null,
        “CoverageType”: null,
        “Insured”: {
          “Identifiers”: [ ],
          “LastName”: null,
          “MiddleName”: null,
          “FirstName”: null,
          “SSN”: null,
          “Relationship”: null,
          “DOB”: null,
          “Sex”: null,
          “Address”: {
            “StreetAddress”: null,
            “City”: null,
            “State”: null,
            “ZIP”: null,
            “County”: null,
            “Country”: null
          }
        }
      }
    ],
    “Guarantor”: {
      “Number”: “10001910”,
      “FirstName”: “Kent”,
      “MiddleName”: null,
      “LastName”: “Bixby”,
      “SSN”: null,
      “DOB”: null,
      “Sex”: null,
      “Spouse”: {
        “FirstName”: “Barbara”,
        “LastName”: “Bixby”
      },
      “Address”: {
        “StreetAddress”: “4762 Hickory Street”,
        “City”: “Monroe”,
        “State”: “WI”,
        “ZIP”: “53566”,
        “County”: “Green”,
        “Country”: “USA”
      },
      “PhoneNumber”: {
        “Home”: null,
        “Business”: null,
        “Mobile”: null
      },
      “EmailAddresses”: [ ],
      “Type”: null,
      “RelationToPatient”: “Father”,
      “Employer”: {
        “Name”: “Accelerator Labs”,
        “Address”: {
          “StreetAddress”: “1456 Old Sauk Road”,
          “City”: “Madison”,
          “State”: “WI”,
          “ZIP”: “53719”,
          “County”: “Dane”,
          “Country”: “USA”
        },
        “PhoneNumber”: “+18083451121”
      }
    }
  }
}
```

The following exemplary Python code ingests the above JSON and extracts only relevant information for storage in the patient database 28:

```
def patient_update_handler(body):
  meta=body[‘Meta’]
  patient=body[‘Patient’]
  demographics=patient[‘Demographics']
  #storing these variables locally for better compute
  hash_ID=hashPHI(demographics[‘FirstName’], demographics[‘LastName’], de-
mographics[‘DOB’], meta[‘Source’][‘ID’])
  patientids = {‘pid1’: ‘none’, ‘pid1type’: ‘none’, ‘pid2’: ‘none’, ‘pid2type’: ‘none’, ‘pid3’: ‘none’,
        ‘pid3type’: ‘none’, ‘pid4’: ‘none’, ‘pid4type’: ‘none’, ‘pid5’: ‘none’. ‘pid5type’: ‘none’, }
  c = 1
  try:
    for item in patient[‘Identifiers']:
      pidkey = ‘pid’+str(c)
      pidtypekey = pidkey+“type”
      patientids[pidkey] = item[‘ID’]
      patientids[pidtypekey] = item[‘IDType’]
      c = c+1
  except:
    print(“Was not able to parse patient indentifiers”)
  #Stores up to 5 Identifiers into a table for ease of placing into collection
  diagnoses = “
  for item in patient[‘Diagnoses’]:
    newstring = item[‘Name’]
    diagnoses = diagnoses+”,“+newstring
  #Grabs full collection of Diagnoses information and puts it into a string
  allergies = “
  for item in patient[‘Allergies']:
    newstring = item[‘Name’]
    allergies = allergies+”,“+newstring
  #Grabs full collection of Allergy information and puts it into a string
  url = f‘https://aig.burstig.com/api/ig/PROD_PATIENT_UPDATE’
  payload = json.dumps({
  “COUNT”: c,
  “PATIENT_ID1”: patientids[‘pid1’],
  “PATIENT_ID1TYPE”: patientids[‘pid1type’],
  “PATIENT_ID2”: patientids[‘pid2’],
  “PATIENT_ID2TYPE”: patientids[‘pid2type’],
  “PATIENT_ID3”: patientids[‘pid3’],
  “PATIENT ID3TYPE”: patientids[‘pid3type’],
  “PATIENT_ID4”: patientids[‘pid4’],
  “PATIENT ID4TYPE”: patientids[pid4type’],
  “PATIENT_ID5”: patientids[‘pid5’],
  “PATIENT_ID5TYPE”: patientids[‘pid5type’],
  “HASH_ID”: hash_ID,
  “EVENT_TYPE”: meta[‘EventType’],
  “EVENT_DATE_TIME”: meta[‘EventDateTime’],
  “SOURCE_ID”: meta[‘Source’][‘ID’],
  “SOURCE_NAME”: meta[‘Source’][‘Name’],
  “SEX”: demographics[‘Sex’],
  “RACE”: demographics[‘Race’],
  “MARITAL_STATUS”: demographics[‘MaritalStatus'],
  “STATE”: demographics[‘Address'][‘State’],
  “ZIP”: demographics[‘Address'][‘ZIP’],
  “AGE”: get_age(demographics[‘DOB’]),
  “ISDECEASED”: demographics[‘IsDeceased’],
  “DIAGNOSES”: diagnoses,
  “ALLERGIES”: allergies
  })
  headers = {
    ‘Authorization’: f”Basic {AUTHENTICATION}”,
    ‘Content-Type’: ‘application/json’
    }
  #defines the headers needed to place the data into collection
  response = requests.request(“PUT”, url, headers=headers, data=payload)
  if (demographics[‘Language’]==“null”):
    language=‘en’
  key_response= add_patient_ids(hash_ID, meta[‘Source’][‘ID’], patientids[‘pid1’], patien-
tids[‘pid2’], patientids[‘pid3’], patientids[‘pid4’], patientids[‘pid5’], demographics[‘FirstName’], de-
mographics[‘LastName’], demographics[‘DOB’])
  patient_data_{——}response= add_{——}patient_data(hash_ID, meta[‘Source’][‘ID’], de-
mographics[‘Sex’], demographics[‘Race’], demographics[‘Language’], demographics[‘Marital-
Status’], “NA”, “NA”, “NA”, “NA”, “NA”, meta[‘EventDateTime’])
  proton_patient_response=createProtonPatient(clinicID=meta[‘Source’][‘ID’],patientID=patien-
tids[‘pid1’],mrn=patientids[‘pid1’])
  return (“Patient Update has been added successfully, Upload Status: “+str(response.sta-
tus_code)+”, Key Addition: “+key_response+”, Patient Data: “+patient_data_response+”, Proton
Patient Creation: “+proton_patient_response)
```

In this way, the system 20 can securely receive and store EHR data from many EHR systems 34.

Turning back to FIG. 1, various instruments, IoT devices, and systems 34, 36, 38, 40, 42, 44, 46, 48, 50, 52, 54, 56, 58 may communicate with system 20 where their data are ingested by ingestion module API 22, processed 26, and stored in patient database 28. Data may be received from these devices using various methods commonly used by those having ordinary skill in the art. Some methods may be more appropriate than others according to the capabilities of each device 34, 36, 38, 40, 42, 44, 46, 48, 50, 52, 54, 56, 58. Some exemplary ways to receive data include by FTP, chron job pull, and POST requests.

An IoT (Internet of Things) device is a device that remotely monitors a patient's vital information, activity, movement, environmental conditions, and other health conditions. Examples of IoT devices include a blood pressure monitor, glucometer, pulse oximeter, ECG (electrocardiogram), thermometer, scale, and wearables such as activity trackers, smart watches, and the like.

The various instruments, IoT devices for monitoring patients, and systems of FIG. 1 include,

Voice Processing 40 for assessing, screening, and tracking the presence and severity of a targeted illness or disease, for example CANARY SPEECH (https://www.canaryspeech.com/), which is hereby incorporated by reference;

Gait Analysis 42 for measuring gait and identifying deviations, for example products sold by PROTOKINETICS. (https://www.protokinetics.com/), which is hereby incorporated by reference;

Cognition software 46, for example products sold by CAMBRIDGE BRAIN SCIENCES (https://www.cambridgebrainsciences.com/) and NEUROTRAX (https://www.neurotrax.com/), which are hereby incorporated by reference. Cambridge Brain Sciences is an online cognitive assessment product. The Neurotrax cognitive testing enables clinicians to obtain objective and actionable assessments of patient cognition;

Magnetic Resonance Imaging (MRI) 44 scans and analytics, for example QYNAPSE (https://qynapse.com/) and ICOMETRIX (https://icometrix.com/), which are hereby incorporated by reference. Icometrix uses cloud based AI to assist healthcare professionals in understanding and quantifying a patient's physical brain;

Driving Cognitive Assessments 48, for example DRIVABLE by IMPIRICA (https://impirica.tech/driveable/), which is hereby incorporated by reference. DriveAble Cognitive Assessment Tool is a computer based assessment system that looks at cognitive abilities needed for safe driving;

Eye Tracking 50, for example RIGHTEYE (https://righteye.com/), which is hereby incorporated by reference;

A smart watch 51, for example a watch made by APPLE, GOOGLE, or SAMSUNG may be used to monitor certain conditions. For example, Rune Labs' Software (https://www.runelabs.io/) on an Apple Watch to monitors common Parkinson's Disease symptoms;

Sleep Apnea Testing 52, for example ITAMAR (https://www.itamar-medical.com/), which is hereby incorporated by reference;

Remote Patient Monitoring 54, for example BIOINTELLISENSE (https://biointellisense.com/) and BIOBEAT (https://www.bio-beat.com/), which are hereby incorporated by reference;

Driving Simulator 56, for example DRIVESAFETY (https://drivesafety.com/), which is hereby incorporated by reference;

Balance products 58, for example ZIBRIO (https://www.zibrio.com/), which is hereby incorporated by reference.

Returning to system 20, the system 20 may also comprise an artificial intelligence (AI) advisor 29 to include in the reports personal treatment protocol recommendations that improve patient outcomes. For example, recommendations may include a Disease Modifying Therapy (DMT) or drug that is most likely to impede the progress of neurological disease. These recommendations are made immediately, while the patient is in the office.

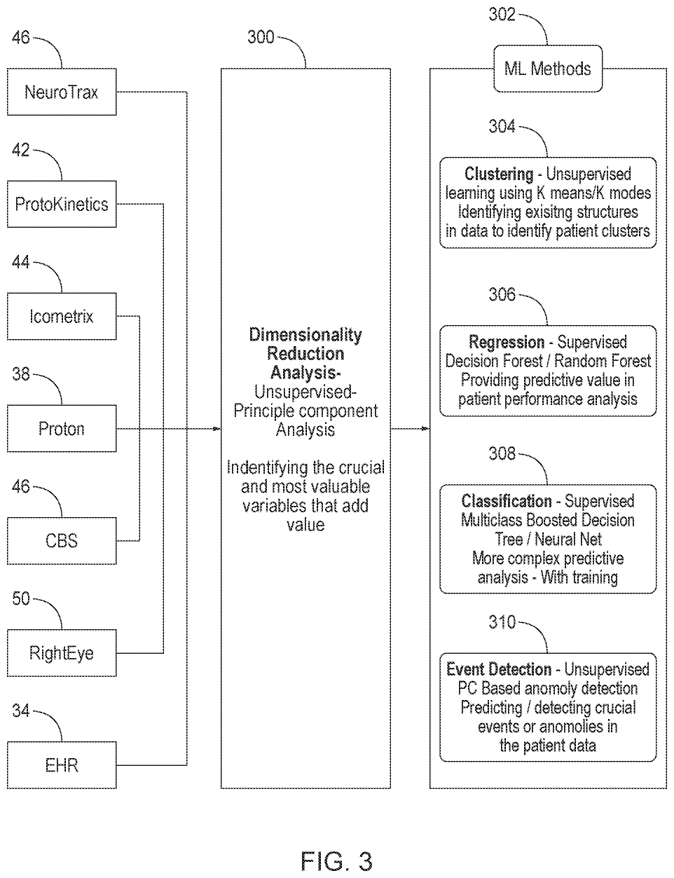

FIG. 3 shows the elements of the AI advisor 29. The Advisor 29 receives data from a plurality of devices disclosed above. The data may be received via the patient database 28, or it may be received directly, or a combination of both. Some of the devices shown are devices 46, 42, 44, 38, 46, 50, and 34. Data from additional devices shown in FIG. 1, or fewer devices, may provide data.

Data is provided to module 300 which performs dimensionality reduction analysis which reduces the number of input variables in the dataset. An unsupervised principal component analysis may be employed to identify the crucial and most valuable variables of the datasets.

Several machine learning methods 302 may be employed to make recommendations such as DMTs and drugs that are more likely to impede the progress of neurological disease. Unsupervised learning using K-means/K-Modes clustering 304 identifies existing structures in data to identify patient clusters. Supervised decision forest/Random forest regression 306 provides predictive value to patient performance data. Supervised multiclass boosted decision tree/neural net classification 308 gives more complex predictive analysis with training. And unsupervised PCA Based anomaly detection event detection 310 predicts and detects crucial events or anomalies in the patient data.

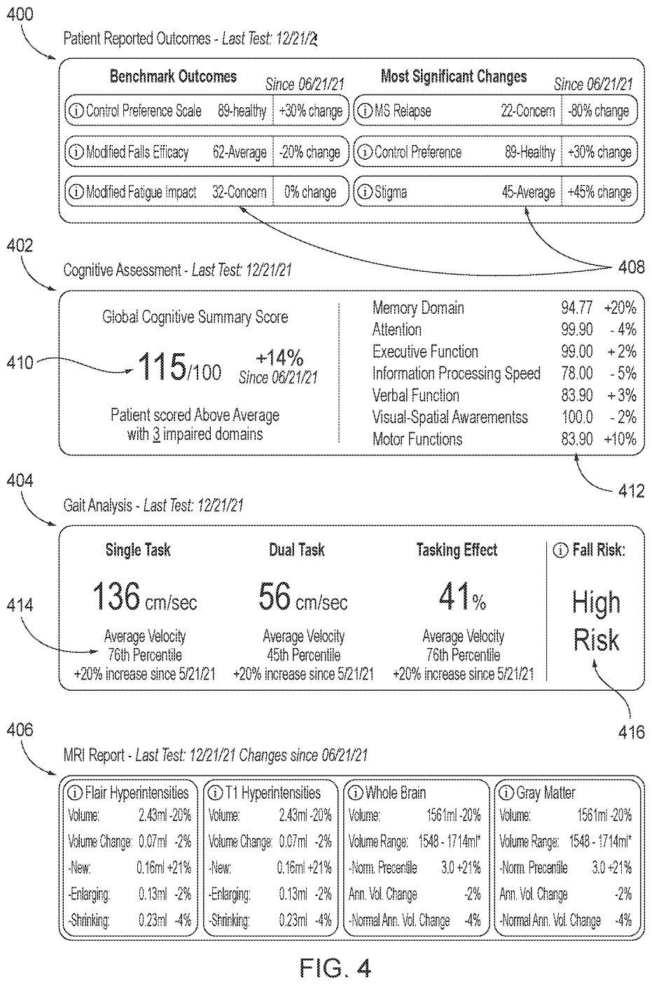

FIG. 4 is an exemplary electronic patient report including data ingested and analyzed as disclosed above. This is an exemplary report, and reports may differ in layout and content according to clinician's preferences and the particular disease being monitored. Furthermore, the report may be dynamic and interactive. For example, a clinician reviewing the report on their device 60 may interact with the report by clicking, or touching in the case of a tablet or phone, different elements of the report to reveal additional information. For example, longitudinal data showing changes over time of the particular item selected may be displayed.

The electronic patient report from the report module may be transmitted to any number of electronic health record systems 34 via the EHR data integrator 36, thereby becoming a part of the patient's official electronic health record.

In this report various data from Patient Report Outcomes 400 are displayed. Note that scores for each PRO are displayed, along with an assessment as to how healthy or concerning the score is, e.g. healthy, average, concern, and so forth. A percent change is since the last test date is also shown.

Similarly a Cognitive Assessment section 402 shows associated scored 410 of key metrics. And, in the Gait Analysis section 404, values of various important measurements are shown along with how that patient is ranked compared to a cross-section of similar patients, e.g. percentile rankings. A fall risk alter 416 is displayed. The MRI Report section 406 similarly shows key measurements and changes since the last test.

Turning back to FIG. 1, the system 20 may also be useful in providing different types of reports for other industries. Pharmaceutical companies 62 may use the data 28, reports 30, and clinical advisor 31 as part of their clinical research in trials. As disclosed above, since data is collected automatically and de-identified, GxP compliance guidelines are ensured. The data, reports, and clinical advisor are useful for Payors 64, such as insurance companies. Because payors are paying fewer claims against a healthier population, payors are improving patient outcomes while simultaneously maximizing financial objectives. Researchers 66 similarly can utilize the data, reports, and clinical advisor to help identify new treatments or improved treatments. And for Regulatory Agencies 68, the system may be used to demonstrate the efficacy of various treatment methods.

As already disclosed, the clinical advisor module 31 generates an interactive dashboard accessible by doctors 60 which displays comprehensive patient information including patient reported outcomes (PROs), digital analytics from devices such as computerized cognitive testing, digital gait analysis, and quantitative MM data, forward looking and predictive analytics, and electronic health record data.

Doctors may interact with the elements displayed on the dashboard to view various aspects of the patient data with varying level of specificity, and in various ways. The system 20 with clinical advisor module 31 enable doctors to validate their clinical decisions and discover new treatment protocol ideas. Additionally, doctors can run “what-if” scenarios to predict the outcome of a slight change in protocol or medicine for a patient. In this way clinicians can determine the best ongoing treatment for their patients. Clinical advisor module 31 provides an objective, evidence-based view of disease trajectories as well as recommendations for the long-term success of therapies.

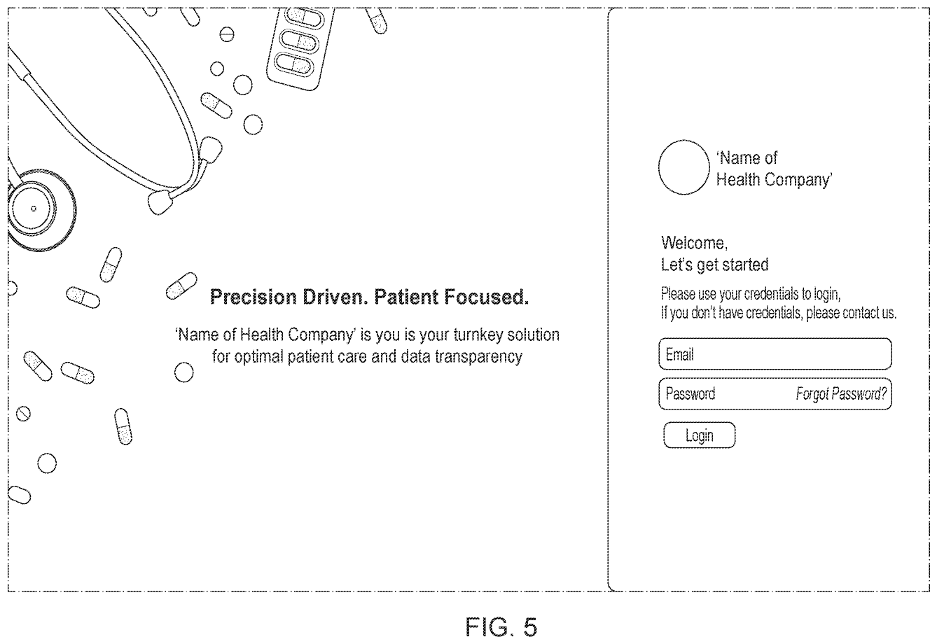

FIGS. 5-11 show various exemplary screenshots of the interactive dashboard created by the clinical advisor module 31. These are just a few exemplary illustrations showing UX (user experience) and UI (user interface) elements that are rendered by the clinical advisor module 31 as part of some exemplary interactive dashboards to communicate highly complex, multidimensional health data, longitudinal health data, cross-sectional health data, scores, statistics, values, measurements, assessments, predictions, and the like. These are just a few examples showing several ways to electronically communicate the patient health data in patient database 28 to doctors 60.

FIG. 5 shows a login screen of an interactive dashboard.

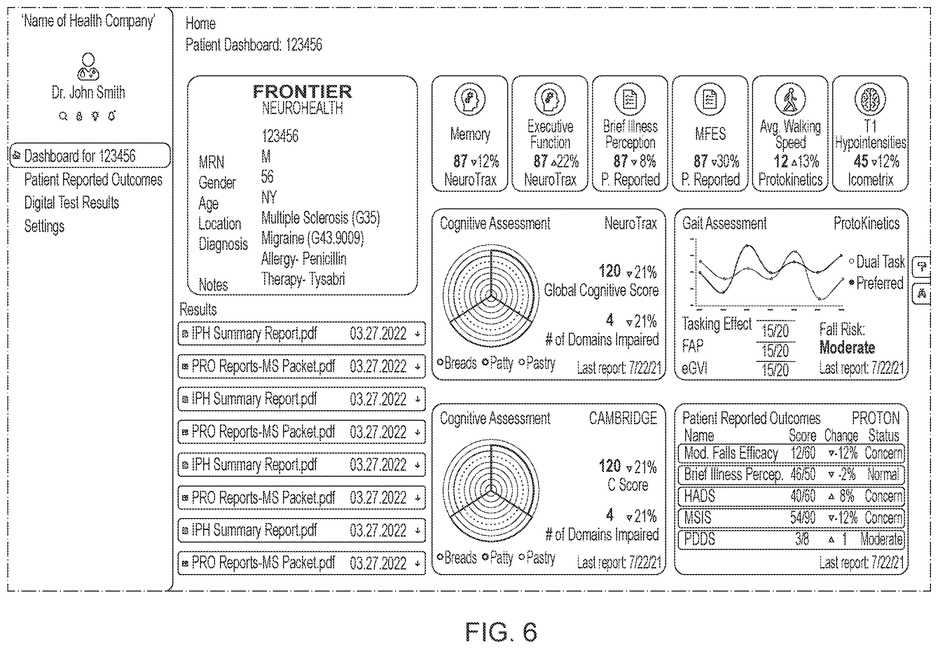

FIG. 6 shows a patient dashboard for an exemplary patient. It comprises many elements to provide an overview of the patient's health. For example, the clinic “Frontier Neurohealth” and essential information about the patient such as name, gender age, location, current diagnosis and notes.

At the top are tiles showing data received from multiple devices (100 of FIG. 1) and processed, stored, and analyzed by system 20. The tiles include Memory (NeuroTrax), Executive Funcition (NeuroTrax), Brief Illness Perception (PRO), MFES (PRO), Average Walking Speed (Protokinetics), and TI Hypointensities (Icometrix). Each tile shows a score and percent change since previous test.

Below the tope tiles are additional tiles portraying more detailed health data received from devices 100 and processed by system 20. These include Cognitive Assessment tiles, a Gait Assessment tile, and a Patient Reported Outcome tile.

All of these tiles can be interacted with. For example a doctor can click on any of the tiles displayed on his computer or tap them if using a tablet or phone to interact with the dashboard, to access deeper insights.

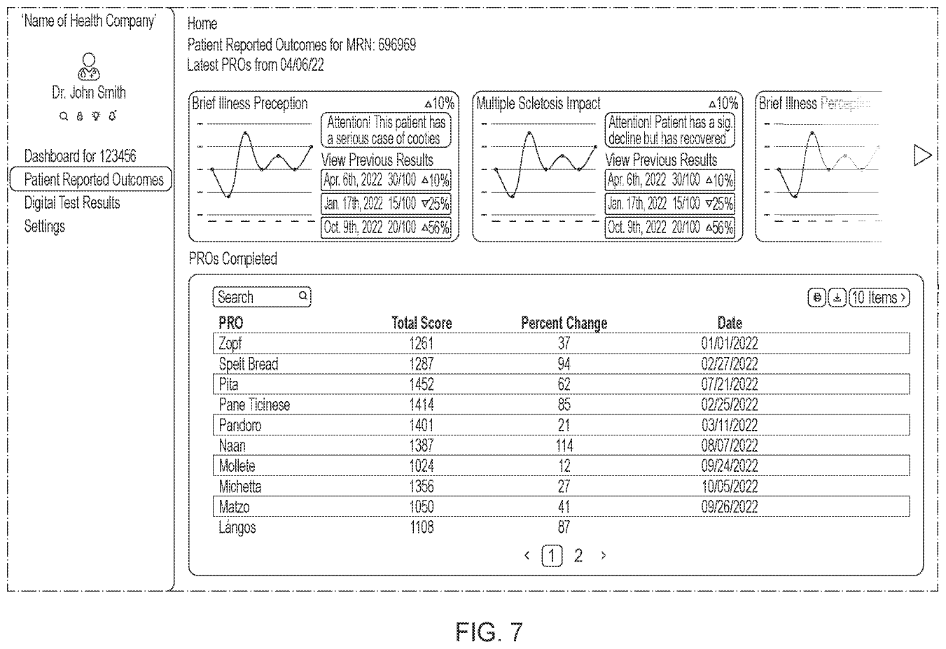

FIG. 7 illustrates another dashboard screen highlighting patient reported outcomes. For example, if the patient reported outcome tile was selected in FIG. 6, a screen like this may appear. At the top are two tiles with a partially faded third and an arrow that can be clicked on to reveal that and more tiles. The tiles (Brief Illness Perception, Multiple Sclerosis Impact Scale) show information about various PROs and longitudinal data for each PRO, represented as a graph. Below tiles is a table summarizing all of the PROs completed by the patient, along with scores, percentage change, and date of completion.

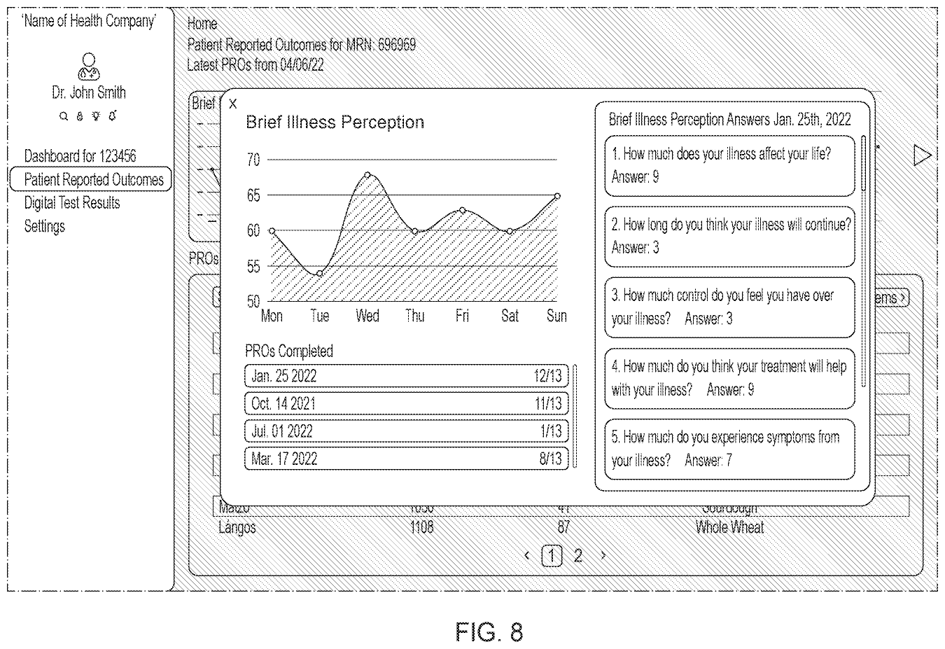

FIG. 8 shows the detailed history of the Brief Illness Perception PRO selected in FIG. 7, along with the actual patient answers. Changes are illustrated in the graph. Each PRO can be selected to view the Brief Illness Perception Answers.

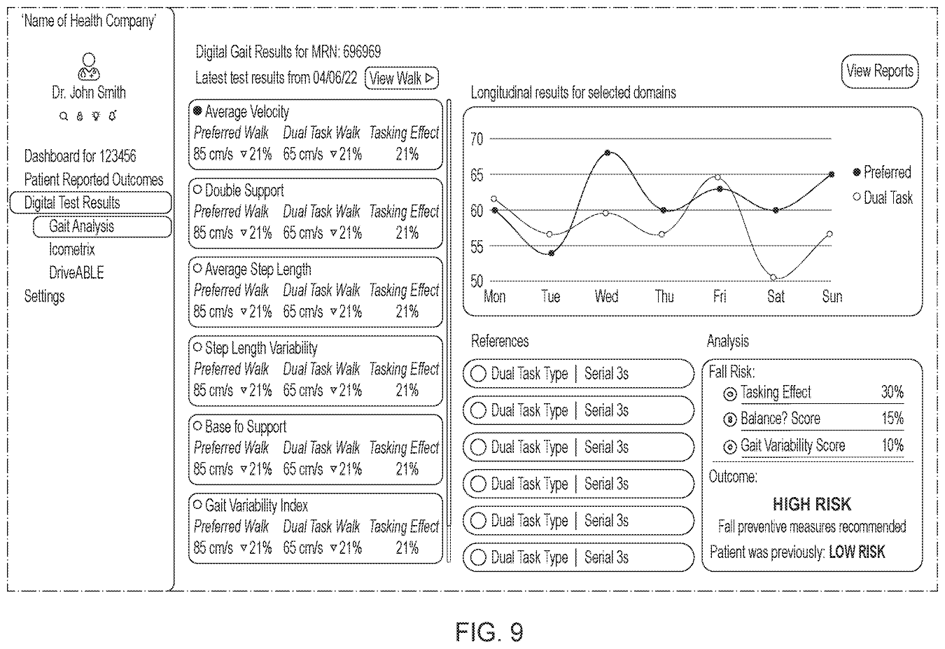

FIG. 9 shows another dashboard rendered as a result of selecting the Gait Assessment tile of FIG. 6 detailing Digital Gait Results obtained, for example, from device 42 of FIG. 1. This dashboard comprehensively and efficiently show actual measurements, changes, longitudinal results, and an Analysis of the data, for example Fall Risk, and Outcome which indicates the patient is at high risk of falling but was previously at low risk.

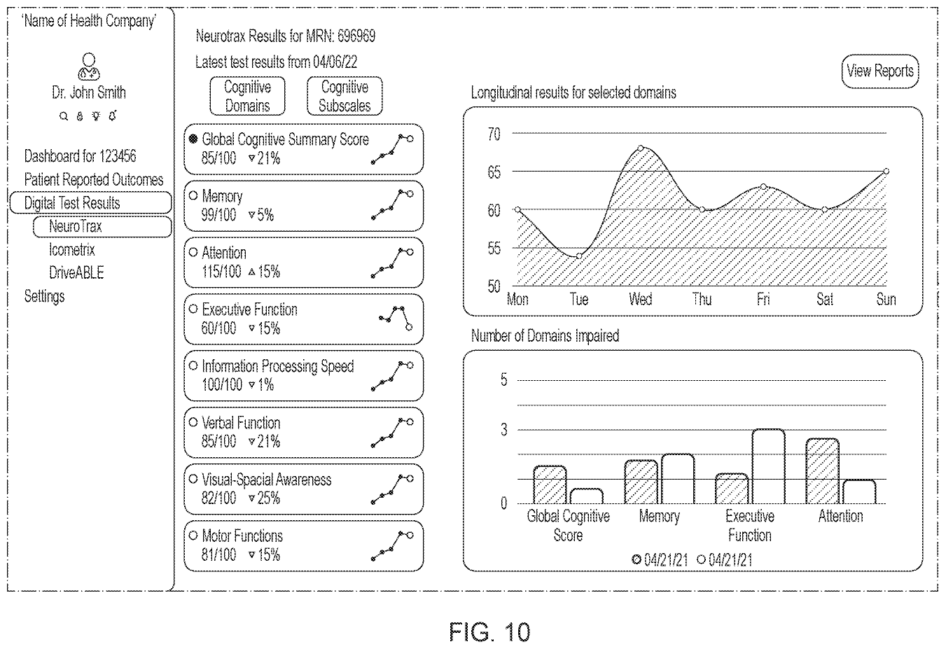

FIG. 10 shows another dashboard of Cognitive Assessment from NeuroTrax, displayed after being selected from the corresponding tile in FIG. 6, graphical depicting various results, value, change in values, and how they have changed over time for the patient. More detailed reports can be generated and views by selecting the “View Report” button icon.

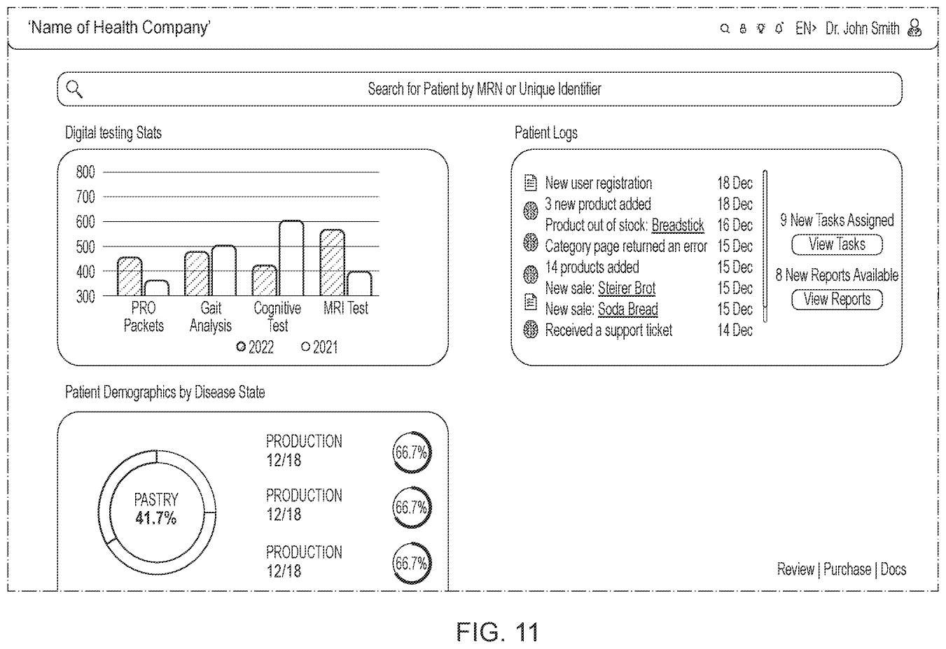

FIG. 11 shows yet another dashboard which illustrates a clinic dashboard. It illustrates, for example, Digital Test Stats, Patient Demographics by Disease State, Patient Logs, New Tasks Assigned, New Reports Available, and has icons to View Tasks and View Reports. It also includes a search bar to search for a patient by MRN or Unique Identifier. The clinic dashboard may display many types information such as, patient logs, medication disbursement, inventory statistics, patient population and demographic statistics, financial performance for the clinic and/or specific providers, overall patient population health metrics, and usage statistics of digital tests.

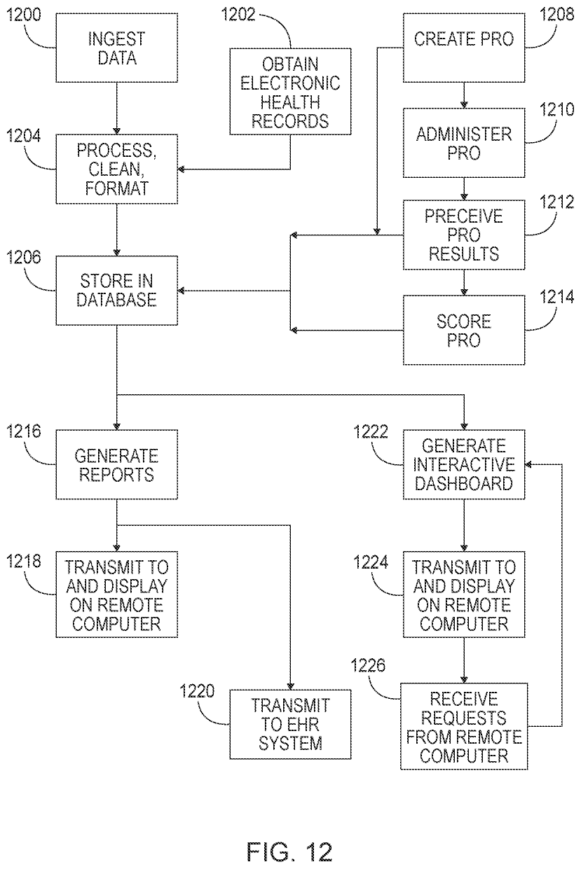

With reference to everything disclosed above, FIG. 12 shows a method for assessing disease progression.

At step 1200, data is ingested from one or more internet connected devices operable to perform a health exam, medical test, or rehabilitative therapy on a patient and provide digital data of the results of the exam, test, or therapy.

At step 1202 digital health records of patients are obtained from electronic health record systems, and storing the records in the patient database.

At step 1206, the ingested data and digital health records are processed, cleaned, and formatted. As disclosed above, the processing, cleaning, and formatting may also include analyzing some or all of the data. Examples of analyzing include identifying trends and changes in the data, executing various analyses algorithms and models on the data such as regression analysis, classification, various from predictive analytics including neural networks and machine learning, clustering models, forecasting models, outliers models, time series models, descriptive analysis, exploratory analysis, inferential analysis, predictive analysis, casual analysis, mechanistic analysis, and any other type of analysis known to those having ordinary skill in the art. Also, the processing, cleaning, and formatting also includes deidentifying health data of patients in the digital health records.

At step 1206, ingested data and digital health records are stored in a patient database. Information and results from analyzing the data in step 1206 may also be stored in the patient database.

At step 1208, creating electronic patient reported outcome questionnaires are created and stored in the patient database.

At step 1210, administering the electronic patient reported outcome questionnaires are administered to a patient on a remote mobile communication device.

At step 1212, receiving from the remote mobile communication device the patient's answers to the questionnaires are received over the internet and stored in the patient database at step 1206.

At step 1214, scoring the patient reported outcome questionnaires are scored, and the scores are stored in the patient database at step 1206.

At step 1218, electronic patient reports are generated from patient data in the patient database, wherein the reports capture a health state of the patient, and the reports are displayed on an internet-connected computing device of a doctor. The reports may be generated automatically, periodically, they may be scheduled, and they may be generated and customized in response to requests from the internet-connected computing device of the doctor.

In the step of generating 1218, the generating may also include performing analytics on some or all of the data. Examples of predictive and other types of analytics include, identifying trends and changes in the data, executing various analyses algorithms and models such as regression analysis, classification, various from predictive analytics including neural networks and machine learning, clustering models, forecasting models, outliers models, time series models, descriptive analysis, exploratory analysis, inferential analysis, predictive analysis, casual analysis, mechanistic analysis, and any other type of analysis known to those having ordinary skill in the art.

At step 1220, the electronic patient reports are optionally transmitted to the electronic health record systems, thereby becoming part of a patient's electronic health record.

At step 1222, an interactive dashboard accessible by an internet-connected computing device of a doctor is generated. At step 1224, the dashboard is transmitted and displayed on the doctor's internet-connected computing device. The doctor may interact with the report through mouse clicks, touching the screen in a case of a phone or tablet, typing in text and data in search boxes and the like, and so forth.

At step 1226, in response to the doctor's interactions with the dashboard, request are receives from the report computer. In response to those requests, at step 1222 the interactive dashboard dynamically modified and generated again, and the process repeats as shown in FIG. 12.

In this loop, displaying on the interactive dashboard in response to requests from the internet-connected computing device of the doctor includes displaying comprehensive patient information representing the health of the patient and progression of disease in the patient over time and in comparison with other similar patients, the patient reported outcome questionnaires and the patient reported outcomes scores.

Also, in this loop, displaying on the interactive dashboard in response to requests representing the selections and interactions with the displayed patient information include displaying specific patient reported outcomes, and the results of the patient's exam, test, or therapy from the one or more internet connected devices operable to perform a health exam, medical test, or rehabilitative therapy on the patient.

Additionally, displaying on the interactive dashboard in response to requests from the internet-connected computing device of the doctor, or in an automated way, may include performing predictive analytics on some or all of the data and displaying the predictive analytics to predict the health outcome of a change in protocol, therapy, or medicine in the treatment of the patient's disease. Examples of predictive and other types of analytics include, identifying trends and changes in the data, executing various analyses algorithms and models such as regression analysis, classification, various from predictive analytics including neural networks and machine learning, clustering models, forecasting models, outliers models, time series models, descriptive analysis, exploratory analysis, inferential analysis, predictive analysis, casual analysis, mechanistic analysis, and any other type of analysis known to those having ordinary skill in the art.

The methods and systems disclosed herein may be implemented on any computer communicating over any network. For example, the computers may include desktop computers, tablets, handheld devices, laptops and mobile devices. The mobile devices may comprise many different types of mobile devices such as cell phones, smart phones, portable computers, tablets, and any other type of mobile device operable to transmit and receive electronic messages.

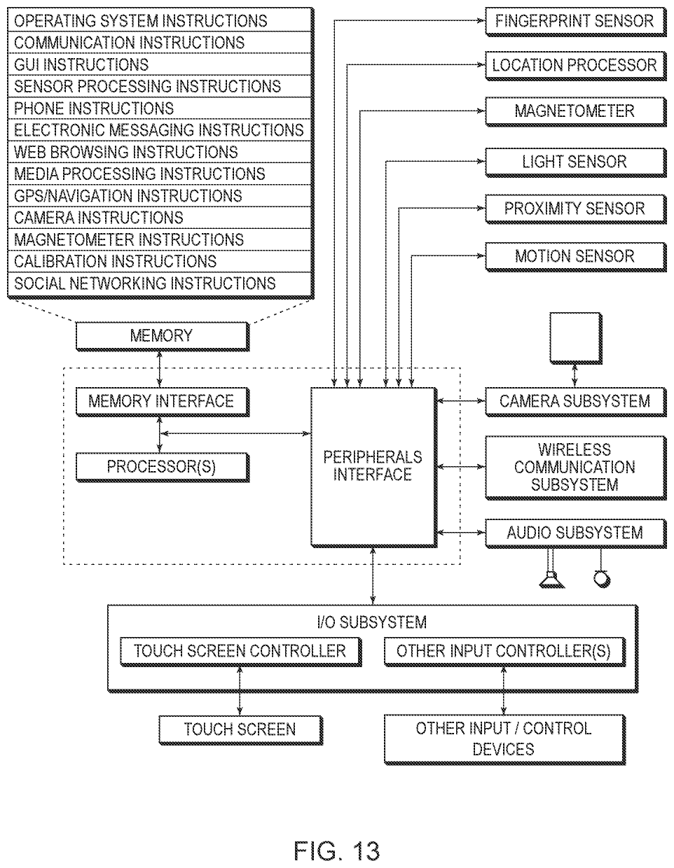

One example of a mobile communication device is a smartphone such as an iPhone or Android phone. Another example of a mobile computing device is a tablet such as a computer tablet such as an iPad, Samsung Galaxy, Microsoft Surface. Other types of mobile communication devices include smart watches and smart glasses. Those skilled in the art will appreciate that there are many types of mobile communication devices compatible with the present invention. FIG. 13 shows the architecture of an exemplary mobile communication device.

While components of certain systems such as system 20 of FIG. 1 are shown together, some of all of the modules of system 20 may be implemented on a cloud computing platform such as on Amazon Cloud Services or Microsoft Azure. In that case, the connections and communications between the various modules are made via a network such as network 10.

The computer network(s) may include the internet and wireless networks such as a mobile phone network. Network work is the internet but may comprise several other interoperable networks. Any reference to a “computer” is understood to include one or more computers operable to communicate with each other. Computers and devices comprise any type of computer capable of storing computer executable code and executing the computer executable code on a microprocessor, and communicating with the communication network(s). For example, a computer may be a web server.

The systems and methods may be implemented on an Intel or Intel compatible based computer running a version of the Linux operating system or running a version of Microsoft Windows, Apple OS, Android, iOS, and other operating systems. Computing devices based on non-Intel processors, such as ARM devices may be used. Various functions of any server, mobile device or, generally, computer may be implemented in hardware and/or in software, including in one or more signal processing and/or application specific integrated circuits.

The computers and, equivalently, mobile devices may include any and all components of a computer such as storage like memory and magnetic storage, interfaces like network interfaces, and microprocessors. For example, a computer comprises some of all of the following: a processor in communication with a memory interface (which may be included as part of the processor package) and in communication with a peripheral interface (which may also be included as part of the processor package); the memory interface is in communication via one or more buses with a memory (which may be included, in whole or in part, as part of the processor package; the peripheral interface is in communication via one or more buses with an input/output (I/O) subsystem; the I/O subsystem may include, for example, a graphic processor or subsystem in communication with a display such as an LCD display, a touch screen controller in communication with a touch sensitive flat screen display (for example, having one or more display components such as LEDs and LCDs including sub-types of LCDS such as IPS, AMOLED, S-IPS, FFS, and any other type of LCD; the I/O subsystem may include other controllers for other I/O devices such as a keyboard; the peripheral interface may be in communication with either directly or by way of the I/O subsystem with a storage controller in communication with a storage device such a hard drive, non-volatile memory, magnetic storage, optical storage, magneto-optical storage, and any other storage device capable of storing data; the peripheral interface may also be in communication via one or more buses with one or more of a location processor such as a GPS and/or radio triangulation system, a magnetometer, a motion sensor, a light sensor, a proximity sensor, a camera system, fingerprint sensor, wireless communication subsystem(s), and audio subsystems.

A non-transitory computer readable medium, such as the memory and/or the storage device(s) includes/stores computer executable code which when executed by the processor of the computer causes the computer to perform a series of steps, processes, or functions. The computer executable code may include, but is not limited to, operating system instructions, communication instruction, GUI (graphical user interface) instructions, sensor processing instructions, phone instructions, electronic messaging instructions, web browsing instructions, media processing instructions, GPS or navigation instructions, camera instructions, magnetometer instructions, calibration instructions, an social networking instructions.

An application programming interface (API) permits the systems and methods to operate with other software platforms such as Salesforce CRM, Google Apps, Facebook, Twitter, Instagram, social networking sites, desktop and server software, web applications, mobile applications, and the like. For example, an interactive messaging system could interface with CRM software and GOOGLE calendar.

A computer program product may include a non-transitory computer readable medium comprising computer readable code which when executed on the computer causes the computer to perform the methods described herein. Databases may comprise any conventional database such as an Oracle database or an SQL database. Multiple databases may be physically separate, logically separate, or combinations thereof.

The features described can be implemented in any digital electronic circuitry, with a combination of digital and analog electronic circuitry, in computer hardware, firmware, software, or in combinations thereof. The features can be implemented in a computer program product tangibly embodied in an information carrier (such as a hard drive, solid state drive, flash memory, RAM, ROM, and the like), e.g., in a machine-readable storage device or in a propagated signal, for execution by a programmable processor; and method steps can be performed by a programmable processor executing a program of instructions to perform functions and methods of the described implementations by operating on input data and generating output(s).

The described features can be implemented in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any type of programming language (e.g., Objective-C, Python, Swift, C#, JavaScript, Rust, Scala, Ruby, GoLang, Kotlin, HTML5, etc.), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. Some elements of a computer are a processor for executing instructions and one or more memories for storing instructions and data. Generally, a computer will also include, or communicate with one or more mass storage devices for storing data files. Exemplary devices include magnetic disks such as internal hard disks and removable disks, magneto-optical disks, and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) for displaying information to the user and a keyboard and a pointing device such as a mouse, trackball, touch pad, or touch screen by which the user can provide input to the computer. The display may be touch sensitive so the user can provide input by touching the screen.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, wired and wireless packetized networks, and the computers and networks forming the Internet.

The foregoing detailed description has discussed only a few of the many forms that this invention can take. It is intended that the foregoing detailed description be understood as an illustration of selected forms that the invention can take and not as a definition of the invention. It is only the claims, including all equivalents, that are intended to define the scope of this invention.

What is claimed is:

1. A system for assessing disease progression comprising:
a patient database for storing health data of patients;
an ingestion module for ingesting data from one or more internet connected devices operable to perform a health exam, medical test, or rehabilitative therapy on a patient and provide digital data of the results of the exam, test, or therapy;
a processing module in communication with the ingestion module and the patient database for processing, cleaning, and formatting the ingested data and writing it into the patient database;
an electronic health record data integrator in communication with the ingestion module for connecting with a plurality of electronic health record systems and obtaining digital health records of patients, and for transmitting electronic patient reports to the plurality of electronic health record systems;
a patient reported outcome (PRO) module in communication with the ingestion module,
for providing an internet-accessible portal that allows clinicians to select and customize electronic patient reported outcome questionnaires via an electronic interface,
for administering the electronic patient reported outcome questionnaires to a patient on a remote mobile communication device, and
for receiving from the remote mobile communication device the patient's answers to the questionnaire over the internet;
a scoring module in communication with the PRO module and the patient database for scoring the patient reported outcome questionnaires;
a report module in communication with the patient database for generating electronic patient reports capturing a health state of the patient and displaying the reports on an internet-connected computing device of a doctor, the report module further in communication with the electronic health record data integrator for transmitting the electronic patient reports to the plurality of electronic health record systems;
an AI advisor module in communication with the patient database and the report module for performing predictive analytics on the health data of patients stored in the patient database; and
a clinical advisor module in communication with the patient database, the report module, and the AI advisor module for generating an interactive dashboard accessible by an internet-connected computing device of a doctor,
which displays on the interactive dashboard in response to requests from the internet-connected computing device of the doctor, comprehensive patient information representing the health of the patient and progression of disease in the patient over time and in comparison with other similar patients, the patient reported outcome questionnaires and patient reported outcomes scores, predictive analytics from the AI advisor module to predict the health outcome of a change in protocol, therapy, or medicine in the treatment of the patient's disease, and
which displays on the interactive dashboard in response to requests representing the selections and interactions with the displayed patient information, specific patient reported outcomes, and the results of the patient's exam, test, or therapy from the one or more internet connected devices operable to perform a health exam, medical test, or rehabilitative therapy on the patient.

2. The invention of claim 1 wherein the ingestion module receives remote patient monitoring data from an IoT device that remotely monitors a patient's vital information, activity, movement, or environmental conditions.

3. The invention of claim 1 wherein the ingestion module receives quantitative MM data.

4. The invention of claim 1 wherein the ingestion module receives digital gait data.

5. The invention of claim 1 wherein the ingestion module receives objective cognitive test data.

6. The invention of claim 1 wherein the ingestion module receives eye movement tracking data.

7. The invention of claim 1 wherein the ingestion module receives at least one of sleep data, voice data, driving data, balance data.

8. The invention of claim 1 wherein the processing module deidentifies health data of patients in the digital health records obtained by the electronic health record data integrator.

9. A method for assessing disease progression comprising the steps of:
(a) ingesting data from one or more internet connected devices operable to perform a health exam, medical test, or rehabilitative therapy on a patient and provide digital data of the results of the exam, test, or therapy;
(b) obtaining digital health records of patients from electronic health record systems, and storing the records in the patient database;
(c) processing, cleaning, and formatting the ingested data and digital health records;
(d) storing the ingested data in a patient database;
(e) creating electronic patient reported outcome questionnaires, and storing the questionnaires in the patient database;
(f) administering the electronic patient reported outcome questionnaires to a patient on a remote mobile communication device;
(g) receiving from the remote mobile communication device the patient's answers to the questionnaires over the internet, and storing them in the patient database;
(h) scoring the patient reported outcome questionnaires, and storing the scores in the patient database;
(i) generating electronic patient reports from patient data in the patient database, wherein the reports capture a health state of the patient, and displaying the reports on an internet-connected computing device of a doctor;
(j) generating an interactive dashboard accessible by an internet-connected computing device of a doctor,
including displaying on the interactive dashboard in response to requests from the internet-connected computing device of the doctor, comprehensive patient information representing the health of the patient and progression of disease in the patient over time and in comparison with other similar patients, the patient reported outcome questionnaires and the patient reported outcomes scores, and
displaying on the interactive dashboard in response to requests representing the selections and interactions with the displayed patient information, specific patient reported outcomes, and the results of the patient's exam, test, or therapy from the one or more internet connected devices operable to perform a health exam, medical test, or rehabilitative therapy on the patient.

10. The method of claim 9 further comprising transmitting the electronic patient reports in (i) to electronic health record systems in (d).

11. The method of claim 9 wherein the step (b) of processing, cleaning, and formatting the ingested data further comprises deidentifying health data of patients in the digital health records.

12. The method of claim 9 further comprising performing analytics on the health data of patients stored in the patient database.

13. The method of claim 12 further comprising displaying on the interactive dashboard in response to requests from the internet-connected computing device of the doctor, predictive analytics to predict the health outcome of a change in protocol, therapy, or medicine in the treatment of the patient's disease.

14. A system for assessing disease progression, the system comprising computer executable code modules stored in a memory of a computer wherein the code modules are executed by a processor of the computer which is in communication with the memory, the memory comprising:
a patient database for storing health data of patients;
an ingestion module for ingesting data from one or more internet connected devices operable to perform a health exam, medical test, or rehabilitative therapy on a patient and provide digital data of the results of the exam, test, or therapy;
a processing module in communication with the ingestion module and the patient database for processing, cleaning, and formatting the ingested data and writing it into the patient database;
an electronic health record data integrator in communication with the ingestion module for connecting with a plurality of electronic health record systems and obtaining digital health records of patients, and for transmitting electronic patient reports to the plurality of electronic health record systems;
a patient reported outcome (PRO) module in communication with the ingestion module,
for providing an internet-accessible portal that allows clinicians to select and customize electronic patient reported outcome questionnaires via an electronic interface,
for administering the electronic patient reported outcome questionnaires to a patient on a remote mobile communication device, and
for receiving from the remote mobile communication device the patient's answers to the questionnaire over the internet;
a scoring module in communication with the PRO module and the patient database for scoring the patient reported outcome questionnaires;
a report module in communication with the patient database for generating electronic patient reports capturing a health state of the patient and displaying the reports on an internet-connected computing device of a doctor, the report module further in communication with the electronic health record data integrator for transmitting the electronic patient reports to the plurality of electronic health record systems; and
a clinical advisor module in communication with the patient database, the report module, and the AI advisor module for generating an interactive dashboard accessible by an internet-connected computing device of a doctor,
which displays on the interactive dashboard in response to requests from the internet-connected computing device of the doctor, comprehensive patient information representing the health of the patient and progression of disease in the patient over time and in comparison with other similar patients, the patient reported outcome questionnaires and patient reported outcomes scores, and
which displays on the interactive dashboard in response to requests representing the selections and interactions with the displayed patient information, specific patient reported outcomes, and the results of the patient's exam, test, or therapy from the one or more internet connected devices operable to perform a health exam, medical test, or rehabilitative therapy on the patient.

15. The system of claim 14 wherein the memory further comprises an AI advisor module in communication with the patient database and the report module for performing predictive analytics on the health data of patients stored in the patient database.

16. The system of claim 15 wherein the clinical advisor module displays the analytics from the AI advisor module to predict the health outcome of a change in protocol, therapy, or medicine in the treatment of the patient's disease.

17. A non-transitory computer-readable medium comprising instructions which, when executed by a processor, causes the processor to the perform the steps of:
(a) ingesting data from one or more internet connected devices operable to perform a health exam, medical test, or rehabilitative therapy on a patient and provide digital data of the results of the exam, test, or therapy;
(b) obtaining digital health records of patients from electronic health record systems, and storing the records in the patient database;
(c) processing, cleaning, and formatting the ingested data and digital health records;
(d) storing the ingested data in a patient database;
(e) creating electronic patient reported outcome questionnaires, and storing the questionnaires in the patient database;
(f) administering the electronic patient reported outcome questionnaires to a patient on a remote mobile communication device;
(g) receiving from the remote mobile communication device the patient's answers to the questionnaires over the internet, and storing them in the patient database;
(h) scoring the patient reported outcome questionnaires, and storing the scores in the patient database;
(i) generating electronic patient reports from patient data in the patient database, wherein the reports capture a health state of the patient, and displaying the reports on an internet-connected computing device of a doctor;
(j) generating an interactive dashboard accessible by an internet-connected computing device of a doctor,
(k) including displaying on the interactive dashboard in response to requests from the internet-connected computing device of the doctor, comprehensive patient information representing the health of the patient and progression of disease in the patient over time and in comparison with other similar patients, the patient reported outcome questionnaires and the patient reported outcomes scores, and
(l) displaying on the interactive dashboard in response to requests representing the selections and interactions with the displayed patient information, specific patient reported outcomes, and the results of the patient's exam, test, or therapy from the one or more internet connected devices operable to perform a health exam, medical test, or rehabilitative therapy on the patient.

18. The computer-readable medium of claim 17 further comprising instructions which, when executed by a processor, causes the processor to the perform the step of transmitting the electronic patient reports in (i) to electronic health record systems in (d).

19. The computer-readable medium of claim 17 further wherein the step (b) of processing, cleaning, and formatting the ingested data further comprises further comprises instruction which, when executed by a processor, causes the processor to perform the step of deidentifying health data of patients in the digital health records.

20. The computer-readable medium of claim 17 further comprising instructions which, when executed by a processor, causes the processor to the perform the step of performing analytics on the health data of patients stored in the patient database.

21. The computer-readable medium of claim 20 further comprising instructions which, when executed by a processor, causes the processor to the perform the step of displaying on the interactive dashboard in response to requests from the internet-connected computing device of the doctor, analytics to predict the health outcome of a change in protocol, therapy, or medicine in the treatment of the patient's disease.
